SEQUENCE LISTING (14 sequences)

SEQ ID NO: 1 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 2 - protein, 1263 aa, synthetic construct; identical to SEQ ID NO: 1

SEQ ID NO: 3 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPHKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 4 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMADLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 5 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMADLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 6 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSREYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMADLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 7 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDRKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMADLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 8 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPHKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMADLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 9 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKRGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMADLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 10 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKRSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMADLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 11 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSREYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 12 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDRKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 13 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKRGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 14 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKRSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

(19) United States
(12) Patent Application Publication
(10) Pub. No.: US 2025/0188431 A1
(43) Pub. Date: Jun. 12, 2025

(54) ENGINEERED NUCLEASE AND USE THEREOF

(71) Applicant: QINGDAO KINGAGROOT CHEMICAL COMPOUND CO., LTD., Qingdao, Shandong (CN)

(72) Inventors: Sudong MO, Qingdao, Shandong (CN); Jun CHEN, Qingdao, Shandong (CN); Xiaoru LI, Qingdao, Shandong (CN)

(21) Appl. No.: 18/842,536

(22) PCT Filed: Jan. 30, 2023
(86) PCT No.: PCT/CN2023/073828

§ 371 Date: Aug. 29, 2024

(30) Foreign Application Priority Data
Mar. 10, 2022 (CN) 202210237008.4

(51) Int. Cl. C12N 9/22; C12N 15/11

(52) U.S. Cl. CPC C12N 9/22; C12N 15/111; C12N 2310/20

(57) ABSTRACT

The present disclosure provides an engineered nuclease for editing living cells and the use thereof.

### CROSS-REFERENCE TO RELATED APPLICATIONS

This patent application is a U.S. National Stage of International Patent Application No. PCT/CN2023/073828, filed Jan. 30, 2023, which claims the benefit of Chinese Patent Application No. 202210237008.4, filed Mar. 10, 2022.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 30,650 Byte XML file named “771780_XML,” created on Aug. 28, 2024.

### TECHNICAL FIELD

The present invention relates to engineered nucleases for editing living cells and application thereof.

### BACKGROUND OF THE INVENTION

In the following discussion, certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

The ability to make precise, targeted changes to the genome of living cells has been a long-standing goal in biomedical research and development. Recently, various nucleases have been identified that allow manipulation of gene sequence, and hence gene function. These nucleases include nucleic acid-guided nucleases. The range of target sequences that nucleic acid-guided nucleases can recognize, however, is constrained by the need for a specific protospacer adjacent motif (PAM) to be located near the desired target sequence. PAMs are short nucleotide sequences recognized by a gRNA/nuclease complex, wherein the complex directs editing of a target sequence in a living cell. The precise PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence. Engineering of nucleic acid-guided nucleases may allow for alteration of PAM preference, allow for editing optimization in different organisms and/or alter enzyme fidelity; all changes that may increase the versatility of a specific nucleic acid-guided nuclease for certain editing tasks.

Thus, there is a need in the art of nucleic acid-guided nuclease gene editing for improved nucleases, such as patent CN111511906A, CN113227368A, etc. The engineered nucleases described herein satisfy this need as well.

### BRIEF SUMMARY OF THE INVENTION

This summary is provided to introduce a selection of concepts in a simplified form that are further described below in the Detailed Description of the Invention. This summary is not intended to identify key or essential features of the claimed subject matter, nor is it intended to be used to limit the scope of the claimed subject matter. Other features, details, utilities, and advantages of the claimed subject matter will be apparent from the following written Detailed Description of the Invention including those aspects illustrated in the accompanying drawings and defined in the appended claims.

The present invention provides an engineered nuclease comprising an amino acid sequence having the following mutation compared with the amino acid sequence as set forth in SEQ ID NO: 1: the amino acid at position 169 is mutated from lysine into arginine.

In one specific embodiment, the amino acid sequence also has one or more mutations selected from the following group: the amino acid at position 589 is mutated from asparagine into any other amino acid, preferably histidine; the amino acid at position 535 is mutated from lysine into any other amino acid, preferably arginine; the amino acid at position 563 is mutated from lysine into any other amino acid, preferably arginine; the amino acid at position 601 is mutated from threonine into any other amino acid, preferably arginine; the amino acid at position 624 is mutated from serine into any other amino acid, preferably arginine.

In another specific embodiment, the amino acid sequence further has at least 80%, at least 85%, at least 90%, at least 95%, at least 96%, at least 97%, at least 98%, at least 99% sequence identity to the amino acid sequence as set forth in SEQ ID NO: 1.

The present invention also provides an engineered nuclease comprising an amino acid sequence that has at least 92%, at least 95%, at least 96%, at least 97%, at least 98%, at least 99% or 100% sequence identity to an amino acid sequence selected from the group consisting of SEQ ID NO: 2, SEQ ID NO: 3, SEQ ID NO: 11, SEQ ID NO: 12, SEQ ID NO: 13 and SEQ ID NO: 14.

In another specific embodiment, the engineered nuclease has improved editing activity in yeast compared with the nuclease having the amino acid sequence as set forth in SEQ ID NO: 1.

The present invention also provides an enzyme cocktail comprising one or a combination of two or more of the engineered nucleases.

The present invention also provides a method of modifying a target region in the genome of a cell, the method comprising:
(a) contacting a cell with:
the engineered nuclease;
an engineered guide nucleic acid capable of complexing with the nuclease; and
an editing sequence encoding a nucleic acid complementary to the target region and having a change in sequence relative to the target region; and
(b) allowing the nuclease, guide nucleic acid and editing sequence to create a genome edit in a target region of the genome of the cell.

In one specific embodiment, wherein the engineered guide nucleic acid and the editing sequence are provided as a single nucleic acid.

In another specific embodiment, wherein the single nucleic acid further comprises a mutation in a protospacer adjacent motif (PAM) site.

The present invention also provides a nucleic acid-guided nuclease system comprising:
(a) the engineered nuclease;
(b) an engineered guide nucleic acid capable of complexing with the nuclease; and
(c) an editing sequence having a change in sequence relative to the sequence of a target region in a genome of a cell;
wherein the system results in a genome edit in the target region in the genome of the cell facilitated by the nuclease, the engineered guide nucleic acid, and the editing sequence.

In one specific embodiment, wherein the engineered guide nucleic acid and the editing sequence are provided as a single nucleic acid.

In another specific embodiment, wherein the single nucleic acid further comprises a mutation in a protospacer adjacent motif (PAM) site.

The present invention also provides a composition comprising:
(a) the engineered nuclease; and
(b) an engineered guide nucleic acid capable of complexing with the nuclease, wherein the engineered guide nucleic acid comprises a loop sequence comprises the sequence of UAUU, UUUU, UGUU, UCUU, UCUUU or UAGU.

In one specific embodiment, the engineered guide nucleic acid is a heterologous engineered guide nucleic acid.

In another specific embodiment, the nuclease is encoded by a codon optimized nucleic acid sequence for use in cells from a particular organism.

The present invention also provides a nucleic acid-guided nuclease system comprising:
(a) the engineered nuclease; and
(b) a heterologous engineered guide nucleic acid capable of complexing with the nuclease.

In one specific embodiment, the system further comprises (c) an editing sequence having a change in sequence relative to the sequence of a target region.

In another specific embodiment, the targeting system results in an edit in the target region facilitated by the nuclease, the heterologous engineered guide nucleic acid, and the editing sequence.

In another specific embodiment, the engineered guide nucleic acid comprises a loop sequence which comprises the sequence of UAUU, UUUU, UGUU, UCUU, UCUUU or UAGU.

In another specific embodiment, the nuclease is encoded by a codon optimized nucleic acid sequence for use in cells from a particular organism.

The present invention also provides a kit for gene editing comprising the engineered nuclease.

The present invention also provides a use of the engineered nuclease in preparing preparation or kits for (i) genome editing; (ii) target nucleic acid diagnosis; (iii) disease treatment.

These aspects and other features and advantages of the invention are described below in more detail.

### BRIEF DESCRIPTION OF THE SEVERAL VIEWS OF THE DRAWING(S)

FIG. 1 represents the activation intensity of mutant K169R and wild-type dMad7 (WT) against AbA on TDO/-Trp/-Leu/-Ura plates.

FIG. 2 represents the resistance of each mutant of dMad7 against 3-AT.

FIG. 3 represents the in vitro enzyme activity assay of Mad7 double mutants.

FIG. 4 represents the sequencing results of OsPPO1 gene editing by Mad7 mutants in rice protoplasts.

FIG. 5 represents the sequencing results of OsYSA gene editing by Mad7 mutants in rice protoplasts.

In FIG. 6, the upper panel represents the results of the rice OsGDI1 gene editing by the Mad7-K169R/N589H mutant; the lower panel represents the results of the rice S-OsGDI1 gene editing by the Mad7-K169R/N589H mutant.

FIG. 7 represents the editing efficiency test of Mad7-K169R/N589H in hairy root systems of soybeans.

FIG. 8 represents the sequencing results of zebrafish tyrosinase gene (tyr) editing by Mad7-K169R/N589H.

FIG. 9 represents the sequencing results of porcine SOCS2 gene editing by Mad7-K169R/N589H.

| Sequence No. | Name |
|---|---|
| SEQ ID NO: 1 | Mad7 |
| SEQ ID NO: 2 | Mad7-K169R |
| SEQ ID NO: 3 | Mad7-K169R/N589H |
| SEQ ID NO: 4 | dMad7 |
| SEQ ID NO: 5 | dMad7-K169R |
| SEQ ID NO: 6 | dMad7-K169R/K535R |
| SEQ ID NO: 7 | dMad7-K169R/K563R |
| SEQ ID NO: 8 | dMad7-K169R/N589H |
| SEQ ID NO: 9 | dMad7-K169R/T601R |
| SEQ ID NO: 10 | dMad7-K169R/S624R |
| SEQ ID NO: 11 | Mad7-K169R/K535R |
| SEQ ID NO: 12 | Mad7-K169R/K563R |
| SEQ ID NO: 13 | Mad7-K169R/T601R |
| SEQ ID NO: 14 | Mad7-K169R/S624R |

### DETAILED DESCRIPTION OF THE INVENTION

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities. Moreover, all of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry, biological emulsion generation, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th edition) W. H. Freeman, New York N.Y.; Gait, “Oligonucleotide Synthesis: A Practical Approach” 1984, IRL Press, London; Nelson and Cox (2000), Lehninger, Principles of Biochemistry, 3rd edition., W. H. Freeman Pub., New York, N. Y.; Berg et al. (2002) Biochemistry, 5th Ed., W. H. Freeman Pub., New York, N. Y.; Cell and Tissue Culture: Laboratory Procedures in Biotechnology (Doyle & Griffiths, Eds., John Wiley & Sons 1998); Mammalian Chromosome Engineering-Methods and Protocols (G. Hadlaczky, Ed., Humana Press 2011); Essential Stem Cell Methods, (Lanza and Klimanskaya, Eds., Academic Press 2011), all of which are herein incorporated in their entirety by reference for all purposes. Nuclease-specific techniques can be found in, e.g., Genome Editing and Engineering from TALENs and CRISPRs to Molecular Surgery, Appasani and Church, 2018; and CRISPR: Methods and Protocols, Lindgren and Charpentier, 2015; both of which are herein incorporated in their entirety by reference for all purposes. Basic methods for enzyme engineering may be found in, Enzyme Engineering Methods and Protocols, Samuelson, Ed., 2013; Protein Engineering, Kaumaya, Ed., (2012); and Kaur and Sharma, “Directed Evolution: An Approach to Engineer Enzymes”, Crit. Rev. Biotechnology, 26:165-69 (2006).

Note that as used herein and in the appended claims, the singular forms “a,” “an,” and “the” include plural referents unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, methods and cell populations that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of ordinary skill in the art that the present invention may be practiced without one or more of these specific details. In other instances, features and procedures well known to those skilled in the art have not been described in order to avoid obscuring the invention.

The term “complementary” as used herein refers to Watson-Crick base pairing between nucleotides and specifically refers to nucleotides hydrogen-bonded to one another, with thymine or uracil residues linked to adenine residues by two hydrogen bonds and cytosine and guanine residues linked by three hydrogen bonds. In general, a nucleic acid comprises a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 out of 10, 9 out of 10, or 10 out of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TTAGCTGG-3′.

The term DNA “control sequence” refers collectively to promoter sequences, polyadenylation signals, transcription termination sequences, upstream regulatory domains, origins of replication, internal ribosome entry sites, nuclear localization sequences, enhancers, and the like, which collectively provide the replication, transcription and translation of a coding sequence in a recipient cell. Not all of these types of control sequences need to be present so long as a selected coding sequence is capable of being replicated, transcribed and—for some components—translated in an appropriate host cell.

As used herein the term “donor DNA” or “donor nucleic acid” refers to a nucleic acid that is designed to introduce a DNA sequence modification (insertion, deletion, substitution) into a locus by homologous recombination using a nucleic acid-guided nuclease. For homology-directed repair, a donor DNA must have sufficient homology to the “cut site” or the flanking region of the site to be edited in a genomic target sequence. The length of one or more homologous arms will depend on, e.g., the type and size of the modification being made. In many instances and preferably, a donor DNA will have two regions of sequence homology (e.g., two homologous arms) to the genomic target locus. Preferably, an “insert” region or “DNA sequence modification” region—a nucleic acid modification that is expected to be introduced into a genomic target locus in a cell—will be located between two homologous regions. A DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of a target sequence. A deletion or insertion may be a deletion or insertion of 1, 2, 3, 4, 5, 10, 15, 20, 25, 30, 40, 50, 75, 100, 150, 200, 300, 400, or 500 or more base pairs of a target sequence.

The term “guide nucleic acid” or “guide RNA” or “gRNA” refers to a polynucleotide comprising 1) a guide sequence capable of hybridizing with a genomic target locus, and 2) a scaffold sequence capable of interacting or complexing with a nucleic acid-guided nuclease.

“Homology” or “identity” or “similarity” refers to sequence similarity between two peptides or, more often in the context of the present disclosure, between two nucleic acid molecules. The term “homologous region” or “homologous arm” refers to a region on a donor DNA with a certain degree of homology with the target genomic DNA sequence. Homology can be determined by comparing a position in each sequence which may be aligned for comparison purpose. When a position in the compared sequence is occupied by the same base or amino acid, then the molecules are homologous at that position. A degree of homology between sequences is a function of the number of matching or homologous positions shared by the sequences.

“Operably linked” refers to an arrangement of elements where the components so described are configured to perform their usual function. Thus, a control sequence operably linked to a coding sequence is capable of affecting the transcription, and in some cases, the translation of the coding sequence. A control sequence does not need to be contiguous to a coding sequence so long as the control sequence functions to direct the expression of the coding sequence. Thus, for example, an intervening sequence that does not translate yet transcribe can be present between a promoter sequence and a coding sequence and the promoter sequence can still be considered “operably linked” to the coding sequence. In fact, such sequences need not reside on the same contiguous DNA molecule (i.e., chromosome) and may still have interactions resulting in altered regulation.

A “promoter” or “promoter sequence” is a DNA regulatory region capable of binding RNA polymerase and initiating transcription of a polynucleotide or polypeptide-coding sequence such as messenger RNA, ribosomal RNA, small nuclear RNA or small nucleolar RNA, guide RNA, or any kind of RNA transcribed by any RNA polymerase I, II or III of any class. A promoter may be constitutive or inducible and in some embodiments, particularly many embodiments in which selection is employed, the transcription of at least one component of the nucleic acid-guided nuclease editing system is under the control of an inducible promoter.

As used herein the term “selectable marker” refers to a gene introduced into a cell, which confers a trait suitable for artificial selection. Generally used selectable markers are well-known to those of ordinary skill in the art. Drug selectable markers such as ampicillin/carbenicillin, kanamycin, chloramphenicol, erythromycin, tetracycline, gentamicin, bleomycin, streptomycin, rifampicin, puromycin, hygromycin, blasticidin, and G418 may be employed. In other embodiments, selectable markers include, but are not limited to human nerve growth factor receptor (detected with MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxy uridine); human glutathione S-transferase alpha (GSTA1; conjugates glutathione to the stem cell selective alkylator busulfan; chemoprotective selectable marker in CD34+ cells); CD24 cell surface antigen in hematopoietic stem cells; human CAD gene to confer resistance to N-(phosphonacetyl)-L-aspartate (PALA); human multi-drug resistance-1 (MDR-1; P-glycoprotein surface protein selected by increased drug resistance or enriched by FACS); human CD25 (IL-2a; detectable by Mab-FITC); 06-methylguanine DNA-methyltransferase (MGMT; selectable by carmustine); and cytidine deaminase (CD; selectable by Ara-C). “Selective medium” as used herein refers to cell growth medium to which has been added a chemical compound or biological moiety that selects for or against selectable markers.

The terms “target genomic DNA sequence”, “target sequence”, or “genomic target locus” refer to any locus in vitro or in vivo, or in a nucleic acid (e.g., genome) of a cell or cell population, in which a change of at least one nucleotide is expected using a nucleic acid-guided nuclease editing system. A target sequence can be a genomic locus or extrachromosomal locus.

A “vector” is any of a variety of nucleic acids that comprise a desired sequence or sequences to be delivered to and/or expressed in a cell. A vector is typically composed of DNA, although RNA vectors are also available. Vectors include, but are not limited to, plasmids, fosmids, phagemids, virus genomes, synthetic chromosomes, etc. As used herein, the phrase “engineered vector” comprises a coding sequence for a nuclease to be used in the nucleic acid-guided nuclease systems and methods of the present disclosure. In a bacterial system, an engineered vector may also comprises the 2 Red recombineering system or an equivalent thereto. An engineered vector also typically comprises a selectable marker. As used herein the phrase “editing vector” comprises a donor nucleic acid and a coding sequence for a gRNA, wherein the donor nucleic acid optionally includes an alteration to the target sequence that prevents nuclease from binding at a PAM or spacer in the target sequence after editing has taken place. An editing vector may also comprise a selectable marker and/or a barcode. In some embodiments, an engineered vector and an editing vector may be combined; that is, the contents of the engineered vector may be found on the editing vector. Further, the engineered and editing vectors comprise control sequences operably linked to, e.g., the nuclease-coding sequence, recombineering system coding sequence (if present), donor nucleic acid, guide nucleic acid, and one or more selectable markers.

General editing of nucleic acid-guided nucleases in genome systems

The present disclosure provides an engineered gene editing nuclease comprising various PAM preferences, optimized editing efficiency in different organisms, and/or an altered RNA-guided enzyme fidelity. Although certain engineered nucleases exhibit enhanced efficiency in, e.g., yeast or mammalian cells, they may be used to edit all cell types including, archaeal, prokaryotic, and eukaryotic (e.g., yeast, fungal, plant and animal) cells.

The engineered nuclease variants described herein improve RNA-guided enzyme editing systems in which nucleic acid-guided nucleases (e.g., RNA-guided nucleases) are used to edit specific target regions in an organism's genome. A nucleic acid-guided nuclease complexed with an appropriate synthetic guide nucleic acid in a cell can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby.

The engineered nuclease may be delivered to cells to be edited as a polypeptide; alternatively, a polynucleotide sequence encoding the engineered nuclease is transformed or transfected into the cells to be edited. The polynucleotide sequence encoding the engineered nuclease may be codon optimized for expression in particular cells, such as archaeal, prokaryotic or eukaryotic cells. Eukaryotic cells can be yeast, fungi, algae, plant, animal, or human cells. Eukaryotic cells may be those of or derived from a particular organism, such as a mammal, including but not limited to human, mouse, rat, rabbit, dog, or non-human mammals including non-human primates. The choice of the engineered nuclease to be employed depends on many factors, such as what type of edit is to be made in the target sequence and whether an appropriate PAM is located close to the desired target sequence. The engineered nuclease may be encoded by a DNA sequence on a vector (e.g., the engineered vector) and be under the control of a constitutive or inducible promoter. In some embodiments, the sequence encoding the nuclease is under the control of an inducible promoter, and the inducible promoter may be separate from but the same as an inducible promoter controlling transcription of the guide nucleic acid; that is, a separate inducible promoter may drive the transcription of the nuclease and guide nucleic acid sequences but the two inducible promoters may be the same type of inducible promoter. Alternatively, the inducible promoter controlling expression of the nuclease may be different from the inducible promoter controlling transcription of the guide nucleic acid.

In general, a guide nucleic acid (e.g., gRNA) complexes with a compatible nucleic acid-guided nuclease and can then hybridize with a target sequence, thereby directing the nuclease to the target sequence. In certain aspects, the RNA-guided enzyme editing system may use two separate guide nucleic acid molecules that combine to function as a guide nucleic acid, e.g., a CRISPR RNA (crRNA) and trans-activating CRISPR RNA (tracrRNA). In other aspects—and used with the engineered nuclease described herein—the guide nucleic acid may be a single guide nucleic acid that comprises both the crRNA and tracrRNA sequences. A guide nucleic acid can be DNA or RNA; alternatively, a guide nucleic acid may comprise both DNA and RNA. In some embodiments, a guide nucleic acid may comprise modified or non-naturally occurring nucleotides. In cases where the guide nucleic acid comprises RNA, the gRNA may be encoded by a DNA sequence on a polynucleotide molecule such as a plasmid, linear construct, or the coding sequence may reside within an editing cassette and is under the control of a constitutive promoter, or in some embodiments, under the control of an inducible promoter as described below.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined using any suitable algorithm for sequence alignment. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In the present methods and compositions, the guide nucleic acid typically is provided as a sequence to be expressed by a plasmid or vector and comprises both the guide sequence and the scaffold sequence as a single transcript under the control of a promoter, and in some embodiments, under the control of an inducible promoter. The guide nucleic acid can be engineered to target a desired target sequence by altering the guide sequence so that the guide sequence is complementary to a desired target sequence, thereby allowing hybridization between the guide sequence and the target sequence. In general, to generate an edit in the target sequence, the gRNA/nuclease complex binds to a target sequence as determined by the guide RNA, and the nuclease recognizes a protospacer adjacent motif (PAM) sequence adjacent to the target sequence. The target sequence can be any polynucleotide endogenous or exogenous to a prokaryotic or eukaryotic cell, or any in vitro polynucleotide. For example, the target sequence can be a polynucleotide residing in the nucleus of a eukaryotic cell. A target sequence can be a sequence encoding a gene product (e.g., a protein) or a non-coding sequence (e.g., a regulatory polynucleotide, intron, PAM, or “junk DNA”).

The guide nucleic acid may be part of an editing cassette that encodes the donor nucleic acid, such as described in USPNs 10,240,167, issued 26 Mar. 2019; U.S. Pat. No. 10,266,849, issued 23 Apr. 2019; U.S. Pat. No. 9,982,278, issued 22 Jun. 2018; U.S. Pat. No. 10,351,877, issued 15 Jul. 2019; and U.S. Pat. No. 10,362,422, issued 30 Jul. 2019; and USSNs 16/275,439, filed 14 Feb. 2019; Ser. No. 16/275,465, filed 14 Feb. 2019; Ser. No. 16/550,092, filed 23 Aug. 2019; and Ser. No. 16/552,517, filed 26 Aug. 2019. Alternatively, the guide nucleic acid may not be part of the editing cassette and instead may be encoded on the engineered or editing vector backbone. For example, a sequence encoding a guide nucleic acid can be assembled or inserted into a vector backbone first, followed by insertion of the donor nucleic acid into, e.g., the editing cassette. In other cases, the donor nucleic acid in, e.g., an editing cassette, can be inserted or assembled into a vector backbone first, followed by insertion of the sequence encoding the guide nucleic acid. In yet other cases, the sequences encoding the guide nucleic acid and the donor nucleic acid (for example, inserted into an editing cassette) are simultaneously but separately inserted or assembled into a vector. In yet other embodiments, the sequence encoding the guide nucleic acid and the sequence encoding the donor nucleic acid are both included in the editing cassette.

The target sequence is associated with a PAM, which is a short nucleotide sequence recognized by the gRNA/nuclease complex. The precise PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence. Engineering of the PAM-interacting domain of a nucleic acid-guided nuclease may allow for alteration of PAM specificity, improve fidelity, or decrease fidelity. In certain embodiments, the genome editing of a target sequence both introduces a desired DNA alteration into a target sequence, e.g., the genomic DNA of a cell, and removes a proto-spacer mutation (PAM) region in the target sequence, to mutate or inactivate the proto-spacer mutation (PAM) region in the target sequence. Inactivating the PAM at the target sequence precludes additional editing of the cell genome at that target sequence, e.g., upon subsequent exposure to a nucleic acid-guided nuclease complexed with a synthetic guide nucleic acid in later rounds of editing. Thus, cells having the desired target sequence edit and an altered PAM can be selected using a nucleic acid-guided nuclease complexed with a synthetic guide nucleic acid complementary to the target sequence. Cells that did not undergo the first editing event will be cut, rendering a double-stranded DNA break, and thus will not continue to be viable. The cells containing the desired target sequence edit and PAM alteration will not be cut, as these edited cells no longer contain the necessary PAM site and will continue to grow and propagate.

The range of target sequences that nucleic acid-guided nucleases can recognize is constrained by the need for a specific PAM to be located near the desired target sequence. As a result, it often can be difficult to target edits with the precision that is necessary for genome editing. It has been found that nucleases can recognize some PAMs very well (e.g., canonical PAMs), and other PAMs less well or poorly (e.g., non-canonical PAMs). Because certain engineered nucleases disclosed herein recognize different PAMs, the engineered nucleases increase the number of target sequences that can be targeted for editing; that is, the engineered nucleases decrease the regions of “PAM deserts” in the genome. Thus, the engineered nucleases expand the scope of target sequences that may be edited by increasing the number (variety) of recognized PAM sequences. Moreover, cocktails of engineered nucleases may be delivered to cells so that target sequences adjacent to several different PAMs may be edited in a single editing run.

Another component of the nucleic acid-guided nuclease system is the donor nucleic acid. In some embodiments, the donor nucleic acid is on the same polynucleotide (e.g., editing vector or editing cassette) as the guide nucleic acid and may be (but not necessarily) under the control of the same promoter as the guide nucleic acid (e.g., a single promoter driving the transcription of both the guide nucleic acid and the donor nucleic acid). The donor nucleic acid is designed to serve as a template for homologous recombination with a target sequence nicked or cleaved by the nucleic acid-guided nuclease as a part of the gRNA/nuclease complex. The polynucleotide of a donor nucleic acid may be of any suitable length, such as about or more than about 20, 25, 50, 75, 100, 150, 200, 500, or 1000 nucleotides in length. In certain preferred aspects, the donor nucleic acid can be provided as an oligonucleotide of 20-300 nucleotides, more preferably 50-250 nucleotides. The donor nucleic acid comprises a region that is complementary to a portion of the target sequence (e.g., a homologous arm). When optimally aligned, the donor nucleic acid overlaps with (is complementary to) the target sequence by, e.g., about 20, 25, 30, 35, 40, 50, 60, 70, 80, 90 or more nucleotides. In many embodiments, the donor nucleic acid comprises two homologous arms (regions complementary to the target sequence) at the mutated or different flanking region between the donor nucleic acid and the target template. The donor nucleic acid comprises at least one mutation or alteration compared to the target sequence, such as an insertion, deletion, modification, or any combination thereof compared to the target sequence.

As mentioned previously, often the donor nucleic acid is provided as an editing cassette, which is inserted into a vector backbone where the vector backbone may comprise a promoter driving transcription of the gRNA and the coding sequence of the gRNA, or the vector backbone may comprise a promoter driving the transcription of the gRNA but not the gRNA itself. Moreover, there may be more than one, e.g., two, three, four, or more, guide nucleic acid/donor nucleic acid cassette inserted into an engineered vector, where each guide nucleic acid is under the control of separate different promoters, separate similar promoters, or where all guide nucleic acid/donor nucleic acid pairs are under the control of a single promoter. In some embodiments-such as embodiments where cell selection is employed—the promoter driving transcription of the gRNA and the donor nucleic acid (or driving more than one gRNA/donor nucleic acid pair) is an inducible promoter. Inducible editing is advantageous in that singulated cells can be grown for several to many times of cell doublings before editing is initiated, which increases the probability that cells with edits will survive, as the double-strand cuts caused by active editing are highly toxic to the cells. This toxicity results both in cell death in the edited colonies, as well as a lag in growth for the edited cells that do survive but must repair and recover after editing. However, once the edited cells have a chance to recover, the size of the colonies of the edited cells will eventually catch up to the size of the colonies of unedited cells. See, e.g., USSNs 16/399,988, filed 30 Apr. 2019; Ser. No. 16/454,865 filed 26 Jun. 2019; and Ser. No. 16/540,606, filed 14 Aug. 2019. Further, a guide nucleic acid may be efficaciously directing the edit of more than one donor nucleic acid in an editing cassette; e.g., if the desired edits are close to one another in a target sequence.

In addition to the donor nucleic acid, an editing cassette may comprise one or more primer sites. The primer sites can be used to amplify the editing cassette by using oligonucleotide primers; for example, if the primer sites are located in flanking regions of one or more of the other components of the editing cassette.

Also, as described above, the donor nucleic acid may comprise, in addition to at least one mutation relative to a target sequence, one or more PAM sequence alterations that mutate, delete or inactivate the PAM site in the target sequence. The PAM sequence alteration in the target sequence renders the PAM site “immune” to the nucleic acid-guided nuclease and protects the target sequence from further editing in subsequent rounds of editing if the same nuclease is used.

In addition, the editing cassette may comprise a barcode. A barcode is a unique DNA sequence that corresponds to the donor DNA sequence such that the barcode can identify the edit made to the corresponding target sequence. The barcode typically comprises four or more nucleotides. In some embodiments, the editing cassettes comprise a collection representing donor nucleic acids, e.g., gene-wide or genome-wide libraries of donor nucleic acids. The library of editing cassettes is cloned into vector backbones where, e.g., each different donor nucleic acid is associated with a different barcode.

Additionally, in some embodiments, an expression vector or cassette encoding components of the nucleic acid-guided nuclease system further encodes an engineered nuclease comprising one or more nuclear localization sequences (NLSs), such as about or more than about 1, 2, 3, 4, 5, 6, 7, 8, 9, 10, or more NLSs. In some embodiments, the engineered nuclease comprises NLSs at or near the amino terminus, NLSs at or near the carboxyl terminus, or a combination.

The engineered and editing vectors comprise control sequences operably linked to the component sequences to be transcribed. As stated above, the promoters driving transcription of one or more components of the engineered nuclease editing system may be inducible, and an inducible system is likely employed if selection is to be performed. A number of gene regulation control systems have been developed for controlling expression of genes in plant, microbe, and animal cells (including mammalian cells), including the pL promoter (induced by heat inactivation of the CI857 repressor), the pBAD promoter (induced by adding arabinose to the cell growth medium), and the rhamnose inducible promoter (induced by adding rhamnose to the cell growth medium). Other systems include the tetracycline-controlled transcription activation system (Tet-On/Tet-Off, Clontech, Inc. (Palo Alto, CA); Bujard and Gossen, PNAS, 89 (12): 5547-5551 (1992)), the Lac Switch Inducible system (Wyborski et al., Environ Mol Mutagen, 28 (4): 447-58 (1996); DuCoeur et al., Strategies 5 (3): 70-72 (1992); U.S. Pat. No. 4,833,080), the ecdysone-inducible gene expression system (No et al., PNAS, 93 (8): 3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others.

Typically, performing genome editing in live cells entails transforming cells with the components necessary to perform nucleic acid-guided nuclease editing. For example, the cells may be transformed simultaneously with separate engineered and editing vectors; the cells may already be expressing the engineered nuclease (e.g., the cells may have already been transformed with an engineered vector, or the coding sequence for the engineered nuclease may be stably integrated into the cellular genome) such that only the editing vector needs to be transformed into the cells; or the cells may be transformed with a single vector comprising all components required to perform nucleic acid-guided nuclease genome editing.

A variety of delivery systems can be used to introduce (e.g., transform or transfect) the components of the nucleic acid-guided nuclease editing system into a host cell. These delivery systems include the use of yeast systems, lipofection systems, microinjection systems, biolistic systems, virosomes, liposomes, immunoliposomes, polycations, lipid-nucleic acid conjugates, virions, artificial virions, viral vectors, electroporation, cell-permeable peptides, nanoparticles, nanowires, exosomes. Alternatively, molecular trojan horse liposomes may be used to deliver nucleic acid-guided nuclease components across the blood brain barrier. The use of electroporation is of particular interest, especially the use of flow-through electroporation (either as an independent instrument or as a module in an automated multi-module system) as described in, e.g., USPNs 10,435,717, issued 8 Oct. 2019; and U.S. Pat. No. 10,443,074, issued 15 Oct. 2019; USSNs 16/550,790, filed 26 Aug. 2019; Ser. No. 10/323,258, issued 18 Jun. 2019; and Ser. No. 10/415,058, issued 17 Sep. 2019.

After the cells are transformed with the components necessary to perform nucleic acid-guided nuclease editing, the cells are cultured under conditions that promote editing. For example, if constitutive promoters are used to drive transcription of the engineered nucleases and/or gRNA, the transformed cells need only be cultured in a typical culture medium under typical conditions (e.g., temperature, CO2 atmosphere, etc.). Alternatively, if editing is inducible—e.g., by activating inducible promoters that control transcription of one or more of the components needed for nucleic acid-guided nuclease editing, such as, e.g., transcription of the gRNA, donor DNA, nuclease, or in the case of bacteria, a recombineering system—the cells are subjected to inducing conditions. The engineered nucleases described herein may be used in automated systems, such as those described in USPNs 10,253,316, issued 9 Apr. 2019; U.S. Pat. No. 10,329,559, issued 25 Jun. 2019; U.S. Pat. No. 10,323,242, issued 18 Jun. 2019; and U.S. Pat. No. 10,421,959, issued 24 Sep. 2019; and USPNs 16/412,195, filed 14 May 2019; Ser. No. 16/423,289, filed 28 May 2019; and Ser. No. 16/571,091, filed 14 Sep. 2019.

### INCORPORATION BY REFERENCE

All publications and patent applications mentioned in this specification are herein incorporated by reference to the same extent as if each individual publication or patent application was specifically and individually indicated to be incorporated by reference.

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example 1

Activation test of Mad7 and its mutant K169R in yeast

To test the activation activity of Mad7 in yeast, a fusion protein (the plasmid may simultaneously recover leucine auxotrophs) of a nuclease protein lacking cleavage activity (dMad7) and GAL4 activation domain (AD) was constructed by referring to the yeast one-hybrid experiment. An aureobasidin A (AbA)-resistance gene driven by 7 tetracycline operons and the recovery of Ura auxotrophs were introduced into a Y1H yeast, and a CrRNA expression plasmid (the plasmid may simultaneously recover tryptophan auxotrophs) modified by pGBKT7 was utilized to express CrRNA to guide the binding of the fusion protein, thereby activating the expression of the aureobasidin A-resistance gene (FIG. 1). The nuclease proteins were amplified by polymerase chain reaction with oligonucleotide primers to introduce an SV40 nuclear localization sequence at the N-terminus consisting of the DNA sequence “ATGGCCCCAAAGAAGAAGCGGAAGGTC” corresponding to a protein sequence of “MAPKKKRKV”. The obtained amplified DNA fragment and a linearized screening plasmid were then transformed into E. coli by homologous recombination and the plasmid was extracted and transformed into the yeast mutant Y1H-7×Tet after ensuring the correct plasmid. Colonies of 2 mm comprising the plasmid were selected, dissolved in saline solution, diluted to 10-1, spread on plates containing 0, 200, 400, 800, 1200 (ng/ml) TDO/-Trp/-Leu/-Ura, and cultured at 30° C. in a thermostatic incubator for 3 consecutive days. Since the fusion protein of the CrRNA-guided nuclease can activate AbA expression in the Y1H-7×Tet yeast, thereby the yeast can grow on TDO/-Trp/-Leu/-Ura plates containing a certain concentration of AbA. The growth density of the culture was proportional to the activation of dMad7-CrRNA and binding activity at 7×Tet site. The results of the analysis are shown in FIG. 1. The mutant K169R (SEQ ID NO: 5) exhibited higher binding activity to the target site compared with the wild-type dMad7 (SEQ ID NO: 4).

### Example 2

Testing the activation ability of activated Mad7 mutants utilizing antagonism between histidine and 3-amino-1,2,4-triazole (3-AT) in yeast

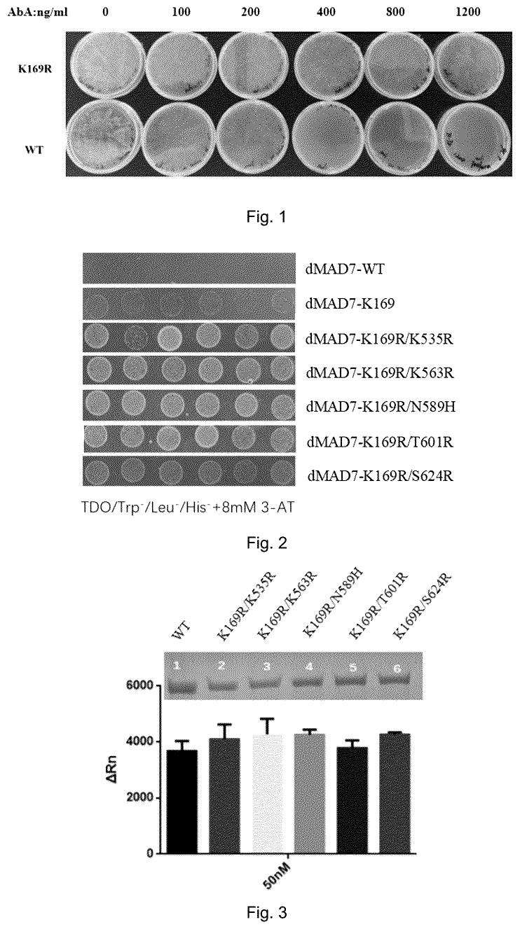

A fusion protein (the plasmid may simultaneously express leucine) of a nuclease protein lacking cleavage activity (dMad7) and GAL4 activation domain (AD) was constructed by referring to the yeast one-hybrid experiment. A promoter of 3 tetracycline operons, histidine gene expression cassette (His) and the recovery of Ura auxotrophs were introduced into a Y1H yeast, and a CrRNA expression plasmid (the plasmid may simultaneously express tryptophan) modified by pGBKT7 was utilized to express CrRNA to guide the binding of the fusion protein, thereby activating the expression of histidine. The nuclease proteins were amplified by polymerase chain reaction with oligonucleotide primers to introduce an SV40 nuclear localization sequence at the N-terminus consisting of the DNA sequence “ATGGCCCCAAAGAAGAAGCGGAAGGTC” corresponding to a protein sequence of “MAPKKKRKV”. The obtained amplified DNA fragment and a linearized screening plasmid were transformed into E. coli by homologous recombination and the plasmid was extracted and transformed into the yeast mutant Y1H-3×Tet after ensuring the correct plasmid. Colonies comprising the plasmid were selected, repeatedly spotted on TDO/Trp-/Leu-/His-+3-AT plates, and cultured at 30° C. in a thermostatic incubator for 3 consecutive days. Since the fusion protein of the CrRNA-guided nuclease can activate His expression in the Y1H-3×Tet yeast, thereby the yeast can grow on TDO/Trp-/Leu-/His-plates containing a certain concentration of 3-AT. The 3-AT resistance of the yeast was in positive correlation with the binding activity between the dMad7 mutant and CrRNA. The results of the analysis are shown in FIG. 2. The mutant K169R (SEQ ID NO: 5), K169R/K535R (SEQ ID NO: 6), K169R/K563R (SEQ ID NO: 7), K169R/N589H (SEQ ID NO: 8), K169R/T601R (SEQ ID NO: 9), K169R/S624R (SEQ ID NO: 10) exhibited higher binding activity to CrRNA compared with the wild-type dMad7 (SEQ ID NO: 4).

### Example 3

In vitro enzyme activity assay of Mad7 mutant proteins

Mad7 double mutant proteins K169R/K535R, K169R/K563R, K169R/N589H, K169R/T601R, K169R/S624R were expressed by bacteria, and CrRNA, and substrate DNA comprising the target site were obtained via in vitro synthesis. The purified protein was first incubated with the synthesized CrRNA to form a RNA-protease complex (RNP), then the cleavage activity of the RNP was activated using the double-stranded DNA comprising the target site as the substrate, and then free short chain single-stranded DNA comprising a fluorophore and quenching group was cleaved. If the single strand was cut, then the quenched fluorophore would be released and the fluorescence intensity would increase. The activities of MAD7 and its mutants were reflected by measuring the fluorescence increment (ΔRn). Since the purified proteins could not be quantified accurately, the amount of wild-type proteins was made greater than or equal to the amount of mutant proteins to be compared, and if the fluorescence increment of any mutant measured was greater than that of the wild type, the in vitro cleavage activity of the mutant was superior to that of the wild type. It is shown in FIG. 3 that the activities of Mad7 double mutant proteins K169R/K535R, K169R/K563R, K169R/N589H, K169R/T601R, K169R/S624R were all superior to those of the wild type.

### Example 4

In vivo editing test of Mad7 mutant proteins in bacteria

Galactose metabolic pathway exists in bacteria: Galactose is phosphorylated by galactokinase (galK, Gene ID: 66670972) to eventually form glucose 6-phosphate which is the substrate of glycolysis and metabolized into pyruvic acid. In the case of sufficient oxygen, it will further enter the tricarboxylic acid cycle to produce a large amount of acidic substances. In the presence of neutral red, the acidic substances can turn it red, so that the knockout can be determined by color. A λ phage protein was introduced to enable bacteria to obtain homologous recombination ability, and the galK homologous knockout fragment was connected to the vector comprising the λ protein. The plasmid was extracted and transformed into E. coli W3110 for propagation, so that the E. coli had a large number of recombinant fragments, and afterwards, were prepared as competent cells and then transformed with Mad7/Mad7 mutant-CrRNA plasmid. The Mad7 protein and 2 protein were produced simultaneously through induction, and in the presence of a large number of homologous recombinant fragments, knockout occurred easily. Therefore, the clones on plates were picked into liquid medium containing neutral red for culture. After culture, those turning turbid yellow were knockout strains, those turning turbid red were non-knockout strains, and those appearing clear yellow were dead strains. Representative data are as shown in Table 1. The knockout efficiency of each protein was calculated according to the following formula after culture:

$$\frac{Turbid yellow ( number )}{Turbid yellow ( number ) + Turbid red ( number )} .$$

As can be seen from Table 1, Mad7 double mutants Mad7-K169R/N589H and Mad7-K169R/S624R had higher knockout efficiencies for galK in bacteria than the wild types did.

TABLE 1
Knockout efficiency of Mad7 and its mutants in bacteria

| | Knockout | Wild-type | | |
|---|---|---|---|---|
| | strains | strains | Dead | Knockout |
| | (yellow) | (red) | strains | efficiency |
| Mad7 | 11 | 84 | 1 | 11.58% |
| Mad7-K169R/N589H | 15 | 76 | 5 | 16.48% |
| Mad7-K169R/S624R | 13 | 81 | 2 | 13.83% |

### Example 5

Editing efficiency test of various Mad7 mutants in rice protoplasts

Suitable Mad7 target sites were designed on rice OsPPO1 (LOC4327918) and OsYSA (LOC4333379) genes, and single target editing test vectors for Mad7 and Mad7 mutants (Mad7-K169R and Mad7-K169R/N589H) were constructed, respectively. The sequences of the target sites were: OsPPO1-CrRNA3: tttc aactccagctgctgttagactgt and OsYSA-CrRNA1: tttc acctggtgcccctcccgccgca, respectively.

Plasmid DNA was extracted using the Promega plasmid extraction kit (Midipreps DNA Purification System, Promega, A7640). Rice protoplasts were prepared for PEG mediated transformation of the test vectors, and the transformation method was referred to “Lin et al., 2018 Application of protoplast technology to CRISPR/Cas9 mutagenesis: from single-cell mutation detection to mutant plant regeneration. Plant Biotechnology Journal https://doi.org/10.1111/pbi.12870”.

Protoplast DNA was extracted by the CTAB method, and the editing efficiencies of the target sites were determined by Hi-TOM sequencing. Hi-TOM detection primers were designed for the target sites, and the lengths of the target fragments were 127 bp and 129 bp, respectively.

PPO1-sgRNA3-Hi-TOM-F:
ggagtgagtacggtgtgcccaaggtatcgctgtcaagttg
PPO1-sgRNA3-Hi-TOM-R:
GAGTTGGATGCTGGATGgcagtcaaatagtgtgcaaacatg
YSA-Hi-TOM-F:
GGAGTGAGTACGGTGTGCcagaatcaggtcgacggcatc
YSA-Hi-TOM-R:
GAGTTGGATGCTGGATGGgacctcatgaaggtgctcgtc

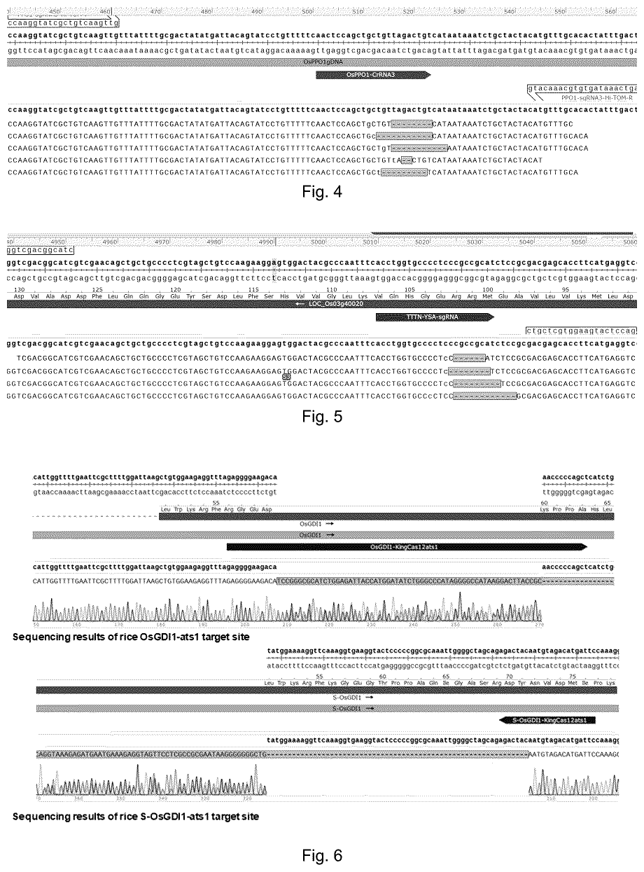

Hi-TOM sequencing analysis was performed on the amplified target fragments and the representative sequencing results are shown in FIG. 4 and FIG. 5. The editing results were respectively statistically analyzed and showed that for rice OsPPO1 target site, the editing efficiency of Mad7-K169R/N589H was 4.58%, the editing efficiency of Mad7-K169R was 2.62% and the editing efficiency of the wild-type Mad7 was 2.16%; for rice OsYSA target site, the editing efficiency of Mad7-K169R/N589H was 4.32%, the editing efficiency of Mad7-K169R was 2.80%, and the editing efficiency of the wild-type Mad7 was 2.10%. Both Mad7-K169R and Mad7-K169R/N589H had higher editing efficiencies than Mad7 did.

### Example 6

Editing test of Mad7-K169R/N589H on rice genes

To obtain rice materials resistant to rice black-streaked dwarf virus (RBSDV), two genes, OsGDI1 (Os05g0418000) and S-OsGDI1 (Os07g0271000), down regulating RBSDV were selected and knocked out using Mad7-K169R/N589H as the nuclease for knockout. “TTTN” was selected as the PAM, and the knockout target sites, OsGDI1-ats1 and S-OsGDI1-ats1, were designed in the third exon of the two genes. The corresponding knockout vector was constructed and rice tissues were genetically transformed using the Agrobacterium transformation method which is commonly used in the art.

The total DNA of the obtained rice TO generation plants was extracted using the CTAB method, and the fragments near the target sites of OsGDI1 and S-OsGDI1 were respectively amplified by PCR. The amplified fragments of each individual plant were sent to Beijing Tsingke Biotechnology Co., Ltd. for testing, and knockouts of the target genes were confirmed according to the sequencing results (as shown in FIG. 6).

The knockout efficiencies of TO transformed seedlings were statistically analyzed. Among rice plants of which target site OsGDI1-ats1 was edited, 27 out of 39 tested rice plants were found to have knockouts, with a knockout efficiency of 69.2%. And for S-OsGDI1-ats1, 22 out of 40 tested rice plants were found to have knockouts, with a knockout efficiency of 55.0%.

### Example 7

Editing efficiency test of Mad7-K169R/N589H in soybean hairy root systems

According to conventional techniques in the art, suitable target sites were designed on soybean FAD (GLYMA_10G286400) and MRP5a (GLYMA_03G056000) genes, and single target editing test vectors for Mad7-K169R/N589H were constructed. And soybean hairy roots were obtained by infecting soybeans with Agrobacterium rhizogenes. The hairy roots with fluorescent markers were taken, and DNA was extracted and then analyzed by high-throughput sequencing analysis.

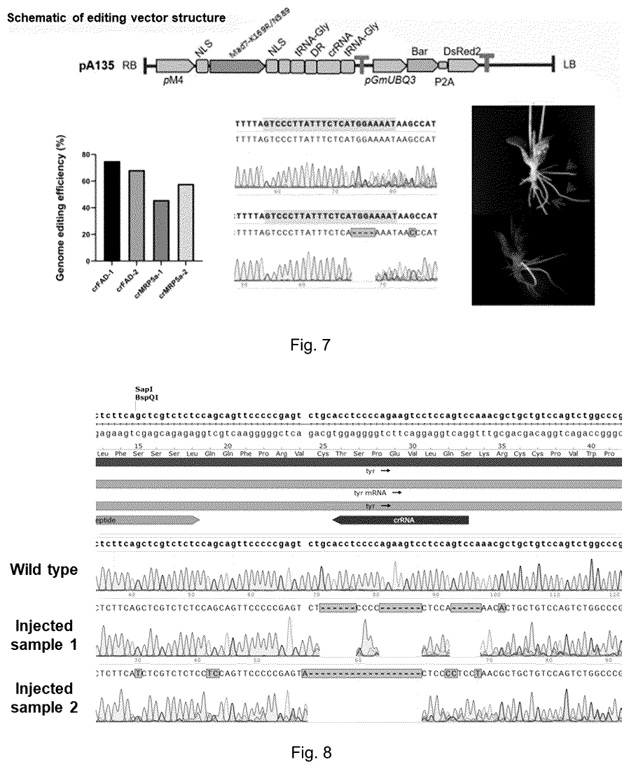

The experimental results are shown in FIG. 7 that Mad7-K169R/N589H produced obvious mutations in the target regions of FAD and MRP5a genes.

### Example 8

Efficiency determination of mutant Mad7-K169R/N589H in zebrafish embryos

To design the target site gactggaggacttctggggaggt of the tyrosinase gene (tyr, Gene ID: 30207) which is the essential gene of zebrafish melanin synthesis pathway, and its PAM sequence was tttg. The corresponding CrRNA chemically synthesized was incubated with the mutant Mad7-K169R/N589H to form a RNA-protease complex (RNP) which was adjusted to a concentration of 1 μM and injected into one-cell embryos of zebrafish. The melanin formation of zebrafish embryos was observed after 48 hours. About 500 living embryos were observed after injections in different batches and 4 embryos were found to have melanin deficiency phenotypes. DNA was extracted from embryos with melanin deficiency. Target sequences were amplified using Dr-TYR-F: GCGTCTCACTCTCCTCGACTCTTC and Dr-TYR-R: GTAGTTTCCGGCGCACTGGCAG, and sequenced.

The sequencing results are shown in FIG. 8 that compared with non-injected wild-type zebrafish embryos, the injected samples both produced definite base deletion mutations at the designed target sites.

### Example 9

Knockout validation of mutant Mad7-K169R/N589H in porcine primary fibroblasts

Target site design for porcine SOCS2 gene (Gene ID: 100037966): Through genome sequence alignment design, target site gggttctcactgacttctaagga was designed at the 5′-UTR of the porcine SOCS2 gene coding sequence, and target site ctaaacacgcctcctgtagegtc was designed after the termination codon of the porcine SOCS2 gene, so as to achieve the purpose of deletion of the SOCS2 gene. The corresponding crRNA chemically synthesized were named CR85 and CR86, respectively.

Porcine fibroblasts (PEF) were RNP transfected using Lipofectamine Stem Transfection Reagent.

TABLE 2
Amount of each component in cell transfection solution

| Component | Amount/Concentration | |
|---|---|---|
| Cell mass | 80% 12 wells | |
| Mad7-K169R/N589H protein | 2 | mg/mL |
| crRNA | 25 | pmol/μL |
| Lipofectamine Stem Transfection Reagent | 4 | μL |
| Total amount of medium (excl. antibodies) | 1 | mL |

The cells were digested 24 h after transfection and counted. The cells were evenly inoculated into individual 10-cm petri dishes with a density of no more than 200 cells/10-cm petri dish, and fresh media were replaced every 48 h. On the 10th day after the cells were divided in dishes, the cells were able to grow into single cell clones of appropriate size, which were digested using cloning cylinders and then transferred to 24-well cell culture plates. After 3-5 days of continuous culture, DNA amplification target sites were extracted from part of cell lines of the single cell clones for sequencing validation.

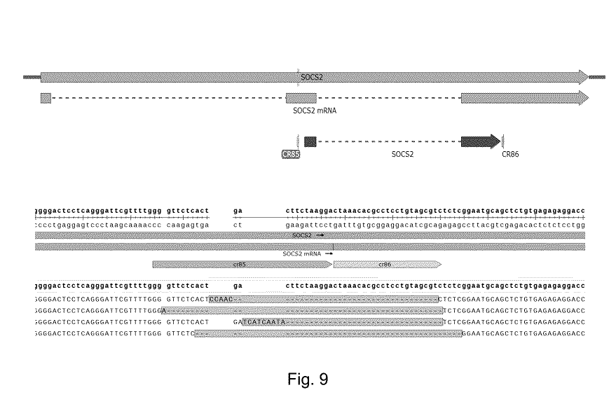

The results are shown in FIG. 9. The sequencing results of multiple cell lines of the single cell clones indicated that definite fragment deletion occurred directly at the designed double target sites.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention.

1. An engineered nuclease comprising an amino acid sequence having the following mutation compared with the amino acid sequence as set forth in SEQ ID NO: 1: the amino acid at position 169 is mutated from lysine into arginine.

2. The engineered nuclease according to claim 1, wherein the amino acid sequence also has one or more mutations selected from the following group: the amino acid at position 589 is mutated from asparagine into any other amino acid, preferably histidine; the amino acid at position 535 is mutated from lysine into any other amino acid, preferably arginine; the amino acid at position 563 is mutated from lysine into any other amino acid, preferably arginine; the amino acid at position 601 is mutated from threonine into any other amino acid, preferably arginine; the amino acid at position 624 is mutated from serine into any other amino acid, preferably arginine.

3. The engineered nuclease according to claim 1 or 2, wherein the amino acid sequence further has at least 80%, at least 85%, at least 90%, at least 95%, at least 96%, at least 97%, at least 98%, at least 99% sequence identity to the amino acid sequence as set forth in SEQ ID NO: 1.

4. The engineered nuclease according to any one of claims 1 to 3 comprising an amino acid sequence that has at least 92%, at least 95%, at least 96%, at least 97%, at least 98%, at least 99% or 100% sequence identity to an amino acid sequence selected from the group consisting of SEQ ID NO: 2, SEQ ID NO: 3, SEQ ID NO: 11, SEQ ID NO: 12, SEQ ID NO: 13 and SEQ ID NO: 14.

5. The engineered nuclease according to any one of claims 1 to 4, having improved editing activity in yeast compared with a nuclease having the amino acid sequence as set forth in SEQ ID NO: 1.

6. An enzyme cocktail comprising one or a combination of two or more of the engineered nucleases according to any one of claims 1 to 5.

7. A method of modifying a target region in the genome of a cell, comprising:
(a) contacting a cell with:
the engineered nuclease according to any one of claims 1 to 5;
an engineered guide nucleic acid capable of complexing with the nuclease; and
an editing sequence encoding a nucleic acid complementary to the target region and having a change in sequence relative to the target region; and
(b) allowing the nuclease, guide nucleic acid and editing sequence to create a genome edit in a target region of the genome of the cell.

8. The method according to claim 7, wherein the engineered guide nucleic acid and the editing sequence are provided as a single nucleic acid.

9. The method according to claim 8, wherein the single nucleic acid further comprises a mutation in a protospacer adjacent motif (PAM) site.

10. A nucleic acid-guided nuclease system comprising:
(a) the engineered nuclease according to any one of claims 1 to 5;
(b) an engineered guide nucleic acid capable of complexing with the nuclease; and
(c) an editing sequence having a change in sequence relative to the sequence of a target region in a genome of a cell;
wherein the system results in a genome edit in the target region in the genome of the cell facilitated by the nuclease, the engineered guide nucleic acid, and the editing sequence.

11. The system according to claim 10, wherein the engineered guide nucleic acid and the editing sequence are provided as a single nucleic acid.

12. The system according to claim 11, wherein the single nucleic acid further comprises a mutation in a protospacer adjacent motif (PAM) site.

13. A composition comprising:
(a) the engineered nuclease according to any one of claims 1 to 5; and
(b) an engineered guide nucleic acid capable of complexing with the nuclease, wherein the engineered guide nucleic acid comprises a loop sequence which comprises the sequence of UAUU, UUUU, UGUU, UCUU, UCUUU or UAGU.

14. The composition according to claim 13, wherein the engineered guide nucleic acid is a heterologous engineered guide nucleic acid.

15. The composition according to claim 13, wherein the nuclease is encoded by a codon optimized nucleic acid sequence for use in cells from a particular organism.

16. A nucleic acid-guided nuclease system comprising:
(a) the engineered nuclease according to any one of claims 1 to 5; and
(b) a heterologous engineered guide nucleic acid capable of complexing with the nuclease.

17. The system according to claim 16, further comprising (c) an editing sequence having a change in sequence relative to the sequence of a target region.

18. The system according to claim 17, wherein the targeting system results in an edit in the target region facilitated by the nuclease, the heterologous engineered guide nucleic acid, and the editing sequence.

19. The system according to claim 16, wherein the engineered guide nucleic acid comprises a loop sequence which comprises the sequence of UAUU, UUUU, UGUU, UCUU, UCUUU or UAGU.

20. The system according to claim 16, wherein the nuclease is encoded by a codon optimized nucleic acid sequence for use in cells from a particular organism.

21. A kit for gene editing comprising the engineered nuclease according to any one of claims 1 to 5.

22. A use of the engineered nuclease according to claims 1 to 5 in the preparation of a formulation or kit, wherein the formulation or kit is used for (i) genome editing; (ii) target nucleic acid diagnosis; (iii) disease treatment.
